(19) United States
(12) Patent Application Publication
(10) Pub. No.: US 2013/0039897 A1
(43) Pub. Date: Feb. 14, 2013

(54) COMPOSITIONS AND METHODS FOR REGULATING NEUTROPHIL MOVEMENT AND NEUTROPHIL NUMBERS IN A BODY REGION

(71) Applicants: Richard Gomer, College Station, TX (US); Jonathan Phillips, Bryan, TX (US); Sarah Herlihy, College Station, TX (US); Anu S. Maharjan, New Britain, CT (US); Darrell Pilling, Cypress, TX (US)

(72) Inventors: Richard Gomer, College Station, TX (US); Jonathan Phillips, Bryan, TX (US); Sarah Herlihy, College Station, TX (US); Anu S. Maharjan, New Britain, CT (US); Darrell Pilling, Cypress, TX (US)

(73) Assignee: The Texas A&M University System, College Station, TX (US)

(21) Appl. No.: 13/571,841

(22) Filed: Aug. 10, 2012

Related U.S. Application Data
(60) Provisional application No. 61/523,015, filed on Aug. 12, 2011.

(51) Int. Cl. A61K 38/48; A61P 11/06; A61P 1/16; A61P 37/06; A61P 25/00; A61P 9/10; A61P 1/00; A61P 1/04; A61P 17/00; A61P 1/18; A61P 19/02; A61P 11/00; A61P 13/12

(52) U.S. Cl. 424/94.3; 424/94.63

(57) ABSTRACT

The disclosure relates to compositions including dipeptidyl peptidase-IV (DPPIV) as well as compositions including an anti-DPPIV antibody operable to bind a DPPIV region structurally homologous to a Dictyostelium autocrine proliferation repressor A (AprA) region. The disclosure also relates to a method of reducing the number of neutrophils in a body region by administering a DPPIV composition to the body region in an amount and for a time sufficient to suppress neutrophil movement into the body region or enhancing neutrophil movement out of the body region. In particular, it relates to a method of reducing the number of neutrophils in a body region suffering from an acute injury or from a chronic or long-term disease. Further, the disclosure relates to a method of increasing the number of neutrophils in a body region by administering an anti-DPPIV antibody operable to bind a DPPIV region structurally homologous to a Dictyostelium AprA region.

### PRIORITY CLAIM

This application claims priority under 35 U.S.C. §119(e) to U.S. Provisional Patent Application Ser. No. 61/523,015, filed Aug. 12, 2011 and titled “Methods of Regulating Neutrophil Movement,” the entirety of which is incorporated by reference herein.

### STATEMENT OF GOVERNMENT INTEREST

At least a portion of this invention was made with government support under Grant No. HL083029 awarded by National Institutes of Health. The United States government has certain rights in the invention.

### TECHNICAL FIELD

The current disclosure relates to methods of regulating neutrophil movement in a patient or neutrophil numbers in a body region. In particular, it relates to methods of regulating neutrophil movement by regulating amounts or activity of the protein dipeptidyl peptidase-IV (“DPPIV”). According to one set of embodiments, neutrophil movement into a body region may be suppressed in a localized manner by providing DPPIV to the region and establishing a DPPIV gradient in the region. In another set of embodiments, neutrophil movement into a body region may be increased by depleting DPPIV or interfering with its function. In an alternative embodiment, increasing local concentration of DPPIV in a body region may facilitate neutrophil movement out of the region.

### BACKGROUND

### Dipeptidyl Peptidase

Dipeptidyl peptidase-IV (“DPPIV”) is a naturally occurring mammalian protein around 100 kDa in size. One known function of DPPIV is as a serine protease enzyme able to remove two amino acids from the N-terminus of a protein if the amino acid next to the amino terminal amino acid is a proline or an alanine DPPIV is active as a dimer in the plasma membrane of lymphocytes and epithelial cells in all organs, where it binds adenosine deaminase, collagen, fibronectin, CD45, glypican 3, and several other proteins. A heavily glycosylated soluble form of DPPIV is also found in the plasma, serum, cerebrospinal fluid, synovial fluid, semen, and urine.

DPPIV is known to play two important roles in the body. First, it degrades glucagon-like peptide-1 (GLP-1), which plays a role in type 2 diabetes. Drugs that block DPPIV's enzymatic activity are also known to block GLP-1 breakdown and thus are used to treat type 2 diabetes. Second, DPPIV affects the activation and inactivation of a number of chemoattractants and other protein factors, such as chemokines, neuropeptides, and vascular regulatory peptides, many of which are involved in tissue remodeling and inflammation.

### Effects of DPPIV Deficiencies

Knock-out mice unable to produce DPPIV have been generated. These mice have normal levels of most leukocytes, but some abnormalities have been observed. In particular, DPPIV knock-out mice have increased severity of experimentally-induced arthritis, with a 2.4 fold increase in the number of cells in the joint. Rheumatoid arthritis patients who have reduced levels of DPPIV in their joints tend to have increased inflammation. Effects of DPPIV have also been observed in the lungs, with DPPIV-deficient rats showing a twofold increase in neutrophils in their lungs as compared to normal rats in ovalbumin-induced lung inflammation.

These effects have been attributed to the enzymatic capabilities of DPPIV, in particular its known effects on chemokines or other chemoattractants.

### Effects on Neutrophils

Neutrophils are immune system cells found in the blood and elsewhere that are involved in the early stages of immune response and inflammation. Neutrophils are typically some of the first cells to leave the blood stream and enter the site of an injury or infection. Neutrophils begin to arrive at an infection or injury site within seconds to minutes, depending on blood circulation to the region. Once there, they release chemicals that further increase the immune or inflammatory response, for instance by recruiting more immune systems cells.

Serious medical conditions can result in patients who have low numbers of neutrophils in their blood stream or whose neutrophils do not appropriately leave the blood stream when needed. Such problems can occur due to genetic disorders, diseases, such as immunological diseases, or the effects of immunosuppressant medications. These patients may be unable to respond appropriately to infection or injury, preventing proper wound healing or allowing infection to set in and become harder to combat later. Accordingly, a need exists for a method of increasing the ability of neutrophils to leave the blood stream and enter injury or infection sites in such patients.

Localized increases in the number of neutrophils in otherwise normal patients may also sometimes be useful. For instance, neutrophils may play a role in combating cancer. Cancer patients with otherwise normal neutrophil levels and activity might benefit from an increased influx of neutrophils to a cancerous tumor or lesion, to the surrounding area, or even to the entire affected organ to help prevent metastasis. Currently, neutrophil-specific treatments are unavailable.

Serious medical conditions may also result when too many neutrophils enter a location in the body. Because the neutrophils have the ability to recruit additional immune system cells and to otherwise enhance the immune and inflammatory responses, they may cause harmful inflammation or damage from the immune system. There are two primary reasons for this to occur, either an immunological disorder or a severe injury.

An overabundance of neutrophils may result from an immunological disorder in any number of ways. For instance, some patients may simply produce too many neutrophils or may produce neutrophils that leave the blood stream too easily. Such patients may be at risk for an over-abundance of neutrophils in even a minor injury. More commonly, the patient may have an autoimmune disease, such as rheumatoid arthritis. Unusually high numbers of neutrophils are found in the joints of patients with rheumatoid arthritis, indicating their role in the development and progression of that disease. Unusually high and harmful numbers of neutrophils may be found in any organ or tissue affected by an autoimmune disease.

Another cause for a harmful overabundance of neutrophils is a severe injury. The body is often not able to address severe injuries and actually causes more harm in attempting to do so. For instance, severe injuries often result in a runaway effect, in which the body responds at higher and higher levels and in more and more ways until the negative effects of the over-response outweigh any positive ones.

For instance, there are over 200,000 cases of acute respiratory distress syndrome (ARDS) in the United States each year. ARDS can result from any severe injury to the lungs, such as infection or inhaling acidic materials, such as vomitus, but it most commonly results from smoke inhalation, particularly during house fires. During ARDS, a large number of lung cells are damaged, causing a rapid influx of neutrophils from the blood stream into the lungs. Once there, the neutrophils release reactive oxygen species and proteases that cause still further damage to lung cells, including the remaining healthy lung cells. These additional damages cause more neutrophils to enter the lungs, resulting in still more damage. Current treatments for ARDS are ineffective at halting this cycle of neutrophil influx and lung damage. As a result, 40% of all patients who develop ARDS die shortly thereafter.

Many ARDS patients do not sustain fatal levels of lung damage during the initial onslaught. As a result, if there were a treatment that, within a matter of a few hours, could deter neutrophils from entering the damaged lung or drive them back out of the damaged tissue, these patients could be saved.

Neutrophils also play a role in chronic obstructive pulmonary disease (COPD). Even though this disease is chronic, rather than acute on nature, there appears to be a constant influx of unhealthy levels of neutrophils into the lung tissue of COPD patients. Furthermore, the amount of neutrophils in the lungs correlates with the severity of the disease. Thus, if the number of neutrophils present in the lungs of COPD patients could be reduced, a corresponding improvement in symptoms is expected.

Treatments able to affect neutrophils entering the lung might also be useful in other situations. For instance, they may be able to help prevent any damaging effects of minor lung injuries, such as inflammation caused by minor air pollution.

Finally, treatments able to deter neutrophils from entering other inappropriate body regions or able to drive them from those regions, such as arthritic joints, may be useful in treating other diseases, like rheumatoid arthritis.

Other problems may result from too few neutrophils in a region of the body. These problems may be corrected or ameliorated by encouraging neutrophil influx.

### SUMMARY

The disclosure is based upon the finding of a new activity of DPPIV as a chemorepellant for neutrophils.

In one aspect the disclosure relates to a composition including DPPIV, a biodegradable polymer bound to the DPPIV, and a pharmaceutically acceptable carrier.

In another aspect, the disclosure relates to a composition including an anti-DPPIV antibody operable to bind a DPPIV region structurally homologous to a Dictyostelium autocrine proliferation repressor A (AprA) region and a pharmaceutically acceptable carrier.

In a third aspect, the disclosure relates to a method of reducing the number of neutrophils in a body region by administering DPPIV composition to the body region in an amount and for a time sufficient to suppress neutrophil movement into the body region or enhancing neutrophil movement out of the body region.

In a fourth aspect, the disclosure relates to a method of reducing the number of neutrophils in the lungs of a patient suffering from ARDS or COPD by administering by inhalation a DPPIV composition to the lungs of the patient in an amount and for a time sufficient to suppress neutrophil movement into the lungs or enhancing neutrophil movement out of the lungs.

In a fifth aspect, the disclosure relates to a method of increasing the number of neutrophils in a body region by administering an anti-DPPIV antibody operable to bind a DPPIV region structurally homologous to a Dictyostelium autocrine proliferation repressor A (AprA) region.

### BRIEF DESCRIPTION OF THE DRAWINGS

A more complete understanding of the present embodiments and advantages thereof may be acquired by referring to the following description taken in conjunction with the accompanying drawings, which depict embodiments of the present disclosure and in which like numbers reflect like features.

FIG. 1A illustrates the normal movement of a neutrophil from the blood stream or extra-body region into a body region.

FIG. 1B illustrates the blocking chemorepellant effect of DPPIV on movement of a neutrophil from the blood stream into a body region.

FIG. 1C illustrates the chemorepellant effect of DPPIV on movement of a neutrophil out of a body into the blood stream or extra-regional space.

FIG. 1D illustrates the effect of removal or inactivation of DPPIV in a tissue to increase movement of neutrophils from the blood stream into the body region.

FIG. 2 illustrates results from an experiment in a Boyden chamber to detect the movement of neutrophils in the presence or absence of recombinant human soluble DPPIV (“rDPPIV”). − indicates buffer and + indicates buffer with 100 ng/ml (1.2 nM) rDPPIV. Values are mean±SEM for at least three independent experiments. * indicates p<0.05 and ** indicates p<0.01 (one-way ANOVA). ‡ indicates p<0.05 (t-test) of raw counts.

FIG. 3A illustrates the results of an experiment in an Insall chamber to detect the migration of neutrophils in the absence of a rDPPIV gradient. Neutrophils were filmed and tracked over 10 minute periods. Red dots represent the average center of mass for all the ending positions of all cells.

FIG. 3B illustrates the results of an experiment in an Insall chamber to detect the migration of neutrophils in the presence of a 0-1.2 nM rDPPIV gradient. Neutrophils were filmed and tracked over 10 minute periods. Orientation is such that the source of rDPPIV is on the left. Red dots represent the average center of mass for all the ending positions of all cells. FIG. 4A illustrates the results of rDPPIV on total cells in the lungs of bleomycin-treated mice. Values are mean±SEM (n=5 for bleomycin, n=4 for saline and bleomycin+DPPIV, n=3 for saline+DPPIV).

FIG. 4B illustrates the results of rDPPIV on neutrophils in the lungs of bleomycin-treated mice. Values are mean±SEM (n=3 for bleomycin and bleomycin+DPPIV, n=2 for saline and saline+DPPIV). * indicates a significant difference with p<0.05 as determined by non-parametric Mann Whitney one-tailed t-test.

FIG. 4C illustrates lung sections of rDPPIV and bleomycin-treated mice stained to show neutrophils. Arrows indicate Ly6G positive cells. Scale bars are 50 gm.

FIG. 4D illustrates the results of neutrophil counts in lung sections of rDPPIV or bleomycin-treated mice. Values are mean±SEM (n=3 for bleomycin and bleomycin+DPPIV, n=2 for saline and saline+DPPIV). ND=none detected.

FIG. 5 illustrates the effects of DPPIV inhibitors in the presence of rDPPIV on neutrophil movement. Values are mean±SEM, n=3. * indicates a significant difference compared to a gradient of DPPIV (p<0.05 by t-test).

### DETAILED DESCRIPTION

The current disclosure relates to DPPIV compositions and methods of use thereof for regulating neutrophil movement into one or more body regions and for limiting the number of neutrophils in one or more body regions by administering DPPIV to the region, and, in some embodiments, to an administration site in the region, in an amount and for a time sufficient to have the desired effect. The disclosure also relates to methods of preventing, alleviating, or avoiding one or more symptoms or complications of an acute injury or chronic or long-term disease characterized by excess neutrophils in one or more body regions. For purposes of this disclosure, a body region may include one or more tissues.

The current disclosure also relates to anti-DPPIV antibody compositions and methods for use thereof to increase neutrophil movement into a body region, or facilitate the retention of neutrophils in that body region.

### Effects of DPPIV

The Dictyostelium autocrine proliferation enzyme repressor A (AprA) protein was recently discovered to function as a chemorepellant of Dictyostelium cells. As a result, mammalian proteins with similar structures, although not necessarily similar sequences, were identified. One such protein, DPPIV, was determined to have a chemorepellant effect on neutrophils. This effect is different from previously identified DPPIV effects in that it is not caused by DPPIV's enzymatic activity on chemoattractants.

In some embodiments, DPPIV may limit the influx of neutrophils into a certain body region or may cause their egress from a region. This may be beneficial in efforts to limit an inflammation response encouraged by neutrophils or other damage resulting from the presence of excessive neutrophils in the body region. For example, in ARDS, neutrophils may cause undesired damage through escalation of an inflammation response. The inhibition of the influx of neutrophils into lung tissue may assist in the treatment of ARDS.

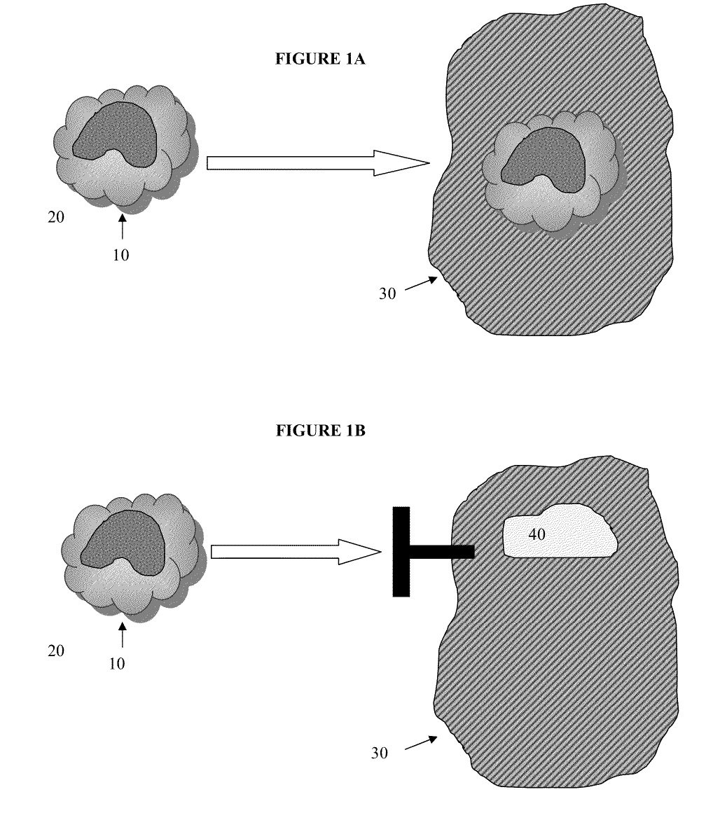

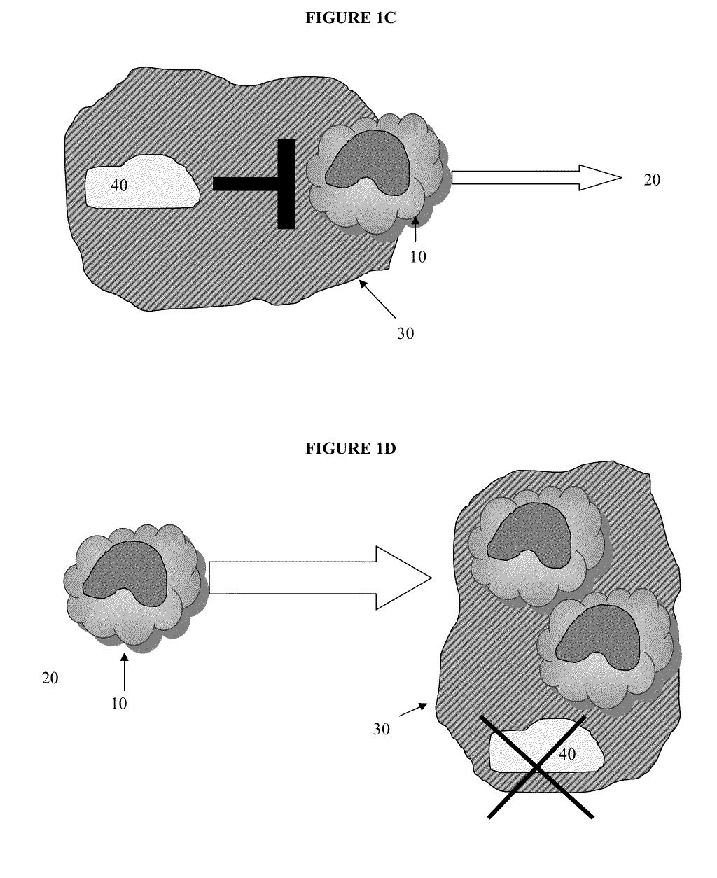

Neutrophils 10 normally move from the blood stream or extra-body region 20 into a body region 30, such as an injured or infected tissue as shown in FIG. 1A. The presence of DPPIV 40 in a region 30 suppresses neutrophil 10 movement, as shown in FIG. 1B. The presence of DPPIV 40 in a region 30 may also force the egress of neutrophils 10 from that region 30 as shown in FIG. 1C. Conversely, the removal or inactivation of DPPIV 40 in a region 30 may increase neutrophil 10 movement into that region 30, as shown in FIG. 1D.

### DPPIV Compositions

Compositions of the current disclosure may include DPPIV in any formulation sufficient to allow its administration to a region of the body. The DPPIV contained in such compositions may be human DPPIV, or it may be a non-human form. It may, in particular, be a mammalian form. It may be from a human or animal source, or it may be recombinant. The DPPIV may be full-length or it may include only portions of a full-length protein. In particular, the DPPIV may include portions of DPPIV that are structurally similar to the Dictyostelium AprA protein, as these structural elements are likely to be responsible for the chemorepellant properties of DPPIV.

Formulations of DPPIV may include any formulation sufficient to preserve the chemorepellant effects of the protein on neutrophils. Different formulations may be useful for different variants of the protein, such as full-length protein or portions thereof, depending on different stabilities. The formulation may also be tailored to the intended use. For instance, the formulation may be suitable for administration via topical administration, injection or inhalation. An inhalable formulation may be suitable for use in a nebulizer. Topical formulations may include any suitable cream, ointment, emollient, gel, foam, or transdermal patch as a carrier.

The form of DPPIV or the formulation may be tailored to retain the DPPIV in a localized fashion. For instance, the formulation may contain materials designed to prevent DPPIV from entering the blood stream. The DPPIV itself may be glycosylated or it may have non-naturally occurring materials, such as polymers, bound to deter its diffusion into the blood stream or away from the site of administration. In particular, biodegradable and non-immunogenic polymers such as polyethylene glycol or poly (amino acids) may be attached to the DPPIV. Any other materials bound to DPPIV may be bound to regions that do not interfere with its chemorepellant effect.

Formulations of DPPIV also include a pharmaceutically acceptable carrier, in particular a carrier suitable for the intended mode of administration, or salts, buffers, or preservatives. In particular, the pharmaceutically acceptable carrier may be tailored to allow DPPIV to retain an active conformation and to avoid degradation.

DPPIV formulations may include other pharmaceutically effective materials, such as other materials able to repel or destroy neutrophils or other immune cells, or other materials able to otherwise induce short or long term beneficial effects in the affected tissue. For example DPPIV formulations may include Serum Amyloid P (SAP), which also affects neutrophils as described in U.S. Provisional Patent Application No. 61/570,445, filed Dec. 14, 2011 and titled “Compositions Associated With and Methods of Managing Neutrophil Movement Using Serum Amyloid P (SAP),” incorporated by reference in material part herein. DPPIV formulations may also include steroids, non-steroid anti-inflammatory drugs (NSAID), or combinations thereof.

### Use of DPPIV

DPPIV, in the form of one of the compositions described above, may be administered locally to a region of the body, for example at one or more administrates sites, in an amount and for a time sufficient to suppress neutrophil movement into that region or to enhance movement of neutrophils out of that region. This results in a decreased number of neutrophils in the region as compared to prior to administration of DPPIV or as compared to the number of neutrophils that would be present absent the administration of DPPIV. The decreased number of neutrophils may prevent, alleviate, or avoid one or more symptoms of complications of an acute injury or chronic or long-term disease characterized by an excess of neutrophils.

The region to which a DPPIV composition is administered may be any body region with an unwanted number of neutrophils and the condition treated may be any acute injury or long-term or chronic disease in which an undesirable number of neutrophils are present.

For example, the body region may be the lungs and the condition may be ARDS or COPD. It may also be asthma or cystic firbrosis. Neutrophils also play a role in asthma, especially in patients with chronic or severe asthma, and asthma resistant to corticosteroids. In addition, it appears that there are increased numbers of neutrophils in non-allergic forms of asthma, such as those induced by air pollution, infection, and obesity. Therefore, in these forms of asthma where conventional therapies are not effective, treatment with DPPIV may be beneficial in these situations.

Cystic fibrosis (CF) is a single-gene disorder caused by mutations in a chloride channel (CFTR). Lung disease is a major problem for patients with CF, leading to persistent bacterial infections and exaggerated inflammatory responses with elevated numbers of neutrophils. These neutrophils appear to be responsible for the release of proteases (enzymes) that damage the lung tissues. Therefore, treatment with DPPIV may be beneficial in these situations.

In another example, the body region may be a joint and the condition may be rheumatoid arthritis.

In addition to treatments associated with the lungs and joints, the features of the present disclosure may also be beneficial in the treatment of other diseases. In some embodiments, the indication being treated may include neutrophil-induced tissue damage. By way of example, the following diseases and regions of the body may be treated within the scope of the present disclosure.

In some embodiments, the present disclosure may be used to treat traumatic brain injury. In addition to the initial insult of the traumatic event to the brain parenchyma, there is a significant amount of secondary damage produced by secondary influx of inflammatory cells and edema. Neutrophil accumulation in the injured brain tissue is an early event seen after traumatic brain injury. Multiple animal models have proposed a role for halting this neutrophil infiltration as a method for limiting secondary damage to injured brain tissue. One of these methods, hypothermia, has been shown to both decrease neutrophil accumulation in an animal model and produce significantly improved clinical outcomes on human patients. Therefore, controlling neutrophil influx may be an effective treatment for brain injury.

The acute phase of tissue transplant rejection may also be treated within the scope of the present disclosure. Acute tissue transplant rejection remains a significant burden in transplant medicine despite improved methods to aid pre-transplant compatibility screening and improved post-transplant immunosuppressive drugs. This rejection is mediated by both allo-antigen primed T-cells that infiltrate the tissue and attract other inflammatory cells such as neutrophils as an effector cell to produce tissue damage and antibody deposition that activates the complement system which also attracts and activates neutrophils. Massive early neutrophil influx into the transplanted tissue has been demonstrated in allografts undergoing acute rejection in both human patients and in animal models of cardiac, liver, kidney, lung, small bowel, and pancreas transplants. Attenuating this inflammatory neutrophilic response may prevent much of the damage done during the acute phase of rejection.

Neutrophil influx into the liver causes liver damage in alcohol-induced neutrophilic steatohepatitis and after acetaminophen overdose. Therefore, these diseases may be treated within the scope of the present disclosure.

In the kidney, neutrophil influx causes tissue damage in acute glomerulonephritis and/or renal inflammation. Acute glomerular injury has been shown to be incited by circulating immune complexes that deposit in the glomerular basement membrane. These immune complexes activate the complement cascade, which then attracts inflammatory cells that further damage the glomerular basement membrane while trying to break down these immune complexes. Neutrophils have been shown to be an important effector cell causing damage to the glomerulus in various forms of acute glomerulonephritis through production of oxidants, cytokines, and chemokines These can include immune-complex type acute glomerulonephritides such as post-streptococcal glomerulonephritis, Goodpasture's syndrome, rapidly progressive glomerulonephritis, membranoproliferative glomerulonephritis, IgA nephropathy, as well as glomerulonephritis caused by antineutrophil cytoplasmic antibody (ANCA)—associated vasculitis (AAV). The early stages of acute glomerulonephritis consist of mainly an inflammatory reaction consisting of neutrophils, with no atrophic or fibrotic changes seen in late stages of chronic glomerulonephritis. This suggests that treating these diseases early in their course with an agent that can prevent neutrophil influx and activation may ameliorate the damage caused by neutrophils during the natural history of the disease. The compositions of the present disclosure may be used to treat early stages of acute glomerulonepthritis.

The compositions of the present disclosure may be used to treat postoperative ileus. Postoperative ileus may be described as a reduction of gastrointestinal motility following an abdominal surgery. It is a significant medical problem that causes patients prolonged discomfort, and it is one of the most common reasons for delayed discharge from the hospital after abdominal surgery. There has not been much progress towards the treatment or prevention of this consequence of surgery, other than the recent adoption of laparoscopic surgery, which has shown to significantly attenuate postoperative ileus, presumably due to a reduced amount of trauma to intra-abdominal tissues. In animal models, it has been shown that trauma to the gastro-intestinal tract is evidenced on a cellular level by an early neutrophilic infiltrate. The invading neutrophils secrete many pro-inflammatory cytokines and cytotoxic substances that contribute to gastrointestinal tract dysmotility. Therefore preventing this early influx of neutrophils may lessen the amount of postoperative ileus, allowing patients to avoid unnecessary suffering and hospitals to save a tremendous amount of resources.

Chronic and long-term intestinal diseases, such as Crohn's disease and ulcerative colitis also involve the influx of neutrophils into an intestinal tissue. The present disclosure includes periodic administration of DPPIV to reduce the number of neutrophils in such tissues and thereby to also reduce one or more symptoms of these diseases.

In some embodiments, the compositions of the present disclosure may be used to treat acute pancreatitis. Acute pancreatitis remains one of the most frequent causes for hospitalization for gastrointestinal related problems, with over 250,000 admissions and costs of over $2 billion per year. Acute pancreatitis can have a wide spectrum of presentations, from mild discomfort to surgical emergencies and multi organ failure resulting in death. From clinical data, it has been demonstrated that pro-inflammatory mediators in the blood of acute pancreatitis patients correlate with the severity of that episode of acute pancreatitis. One of these mediators, PMN-elastase, which is secreted by neutrophils, was found to be one of the most useful indicators of severity. Animal models of acute pancreatitis have also exhibited an early influx of neutrophils into the pancreatic tissue, and that inhibiting this neutrophilic response by ways of neutrophil depletion and neutrophilic receptor blockade have lessened the severity of the disease course of both the pancreatitis itself and associated distant organ damage. Compositions of the present disclosure may also me used to treat chronic pancreatitis.

In the skin, neutrophil influx causes tissue damage in Sweet's syndrome/acute febrile neutrophilic dermatosis, rheumatoid neutrophilic dermatitis, pyoderma gangrenosum, subcorneal pustular dermatosis, Behcet's syndrome, palmoplantar pustulosis, neutrophilic eccrine hidradenitis, bowel-associated dermatosis-arthritis syndrome, and synovitis-acne-pustulosis-hyperostosis osteomyelitis (SAPHO) syndrome.

Neutrophil influx may also cause tissue damage in systemic septic shock, and treatment thereof is within the scope of the present disclosure.

Gout and other crystal-induced arthropathies, which are classic inflammatory diseases where neutrophils cause a considerable amount of damage, may also be treated with formulations of the present disclosure.

Additionally, reperfusion injury, such as pressure ulcers, diabetic foot ulcers, myocardial infarction, stroke, ischemic brain injury, and ischemic bowel disease, may be treated with formulations of the present disclosure. Reperfusion injury occurs following the return of blood flow to a tissue. During the period that blood flow to a tissue is restricted or stopped, the lack of oxygen (ischemia) leads to cell damage and necrosis, which when blood flow returns (reperfusion) leads to the influx of immune cells, led by neutrophils. The neutrophils are then activated by the presence of the dead and dying cells, leading to inflammation. The administration of DPPIV to the local area may lead to the movement of neutrophils away from the site of reperfusion injury, thus reducing tissue damage and preventing further influx of immune cells.

The DPPIV may be supplied to the region in any suitable manner. For instance, in patients with rheumatoid arthritis, it may be injected into an administration site in an affected joint via fine needles. For a patient with danger of an abnormally strong response to a minor injury, it may be applied topically to a minor wound. For patients with ARDS or lung irritation, it may be provided via inhalation, for instance using a nebulizer. For patients with bowel-based neutrophil indications, an ingestible form may be used.

In general, administration techniques may be tailored to contain the DPPIV in a body region. Due to the chemorepellant properties of DPPIV, if it is applied too broadly, it could easily have the unintended effect of forcing neutrophils into the injured or infected tissue. Using ARDS as an example, the DPPIV may be applied via the airways because the injurious agent enters the lungs through the airways. Thus, the DPPIV is applied in the greatest concentration to the regions that are most damaged and where repulsion of neutrophils is most needed. If DPPIV were instead injected into an ARDS patient it would be in its greatest concentration in the blood and would actually force neutrophils from the blood into the lungs, exacerbating the problem.

DPPIV will likely move out from its administration site to form a concentration gradient in a body region. As a result, DPPIV dosage may be adjusted to achieve a desired DPPIV gradient. As noted above, the form of DPPIV, particularly the amount of glycosylation or presence of other diffusion-hampering material, may also be adjusted to achieve the desired concentration gradient. In some uses, such as in rheumatoid arthritis patients, sufficient diffusion from the administration site into the body region may be desirable to allow treatment of a large region, such as an entire joint, with a minimal number of administration sites. In other embodiments, such as in ARDS patients, where the region to be treated is very near a blood supply and essentially the entire affected region is available as a treatment site, the desirable amount of diffusion into the body region may be much more limited.

In some embodiments involving acute neutrophil influx, DPPIV may first be administered within 30 minutes, within 45 minutes, within 60 minutes of a severe injury that leads to an acute influx of neutrophils. In other embodiments, DPPIV may first be administered up to within 24 or even within 48 hours after such an injury. In many embodiments addressing acute neutrophil influx, treatment is ideally begun as soon as possible after the injury, however, not all patients or the cause of their injury are discovered until some time has passed. Treatment may be provided continuously or at intervals until the danger of neutrophil influx passes. For example, in some embodiments, treatment may be provided continuously or at intervals for at least 24 hours, at least 48 hours, at least 72 hours, at least 96 hours, or at least every 120 hours. One may determine when it is appropriate to cease treatments by observing when neutrophil influx, or the symptoms of neutrophil influx, no longer occurs. In embodiments in which treatments are administered at intervals, intervals may be spaced such that substantial neutrophil influx does not resume between treatments or so that a DPPIV gradient is maintained in the in the body region. For example, treatments may be repeated at least every 30 minutes, at least every 60 minutes, at least every 120 minutes, or at least every 24 hours. The time between intervals may increase as time after the injury increases.

In treatment of chronic or long-term diseases, such as rheumatoid arthritis or COPD, treatment may be administered periodically at intervals sufficient to decrease the number of neutrophils in the affected body region. For example, treatment may be administered at least every 24 hours, every 48 hours, every 72 hours, every 96 hours, every 120 hours, every week, or every two weeks.

The amount of DPPIV administered may vary depending on the location of administration, the mode of administration, whether an acute injury or chronic or long term disease is being addressed, the planned treatment regimen, including dosing intervals, and the severity of the injury or disease. The concentration of DPPIV in human blood ranges from 400 to 800 ng/ml, or 4.7 to 9.4 nM. Accordingly, the amount administered will likely have a concentration higher than these amounts.

According to one embodiment, DPPIV may be administered in an amount sufficient to generate a concentration gradient of at least 0.10 ng/ml/μm in the body region treated.

The weight of a single mouse lungs is typically 110-150 mg. In the Examples, 0.9 μg of DPPIV was administered to both mouse lungs and was efficient in repelling neutrophils from the lungs. Thus, administration of between 3 μg to 4 μg of DPPIV per mg of tissue is sufficient to repel neutrophils from that tissue. In alternative embodiments, administration of between 0.005 μg to 0.001 μg of DPPIV per mg of tissue may be used.

Alternatively, in the bleomycin mouse model of ARDS that discussed in the Examples, the average weight of the mice treated with bleomycin and then treated with DPPIV was 22 grams, and one mouse where DPPIV inhibited bleomycin-induced neutrophil influx weighed 27.5 grams. Thus efficacy was observed using 0.041 μg of DPPIV per g of body weight, or 41 μg per kg of body weight, with one mouse showing efficacy at a dose of 33 μg per kg of body weight. In general, DPPIV may be administered in an amount of between 30 μg to 50 μg per kg of body weight.

As noted above, alternative dosages and methods of calculating dosages may be used depending a variety of factors.

### DPPIV Reduction

As explained in the background above, some patients may benefit from increased neutrophil movement into or retention in a location. Such patients therefore may benefit from administration of a DPPIV-inactivator or inhibitor to that region. An inactivator or inhibitor may neutralize the ability of DPPIV to inhibit neutrophil influx or promote neutrophil egress. One such inactivator may be an anti-DPPIV antibody. In particular, it may be an antibody whose antigen lies in a region of DPPIV that is structurally similar to a Dictyostelium AprA protein region. As noted above, such regions are likely responsible for DPPIV's chemorepellant activities, such that their blockage by an antibody is expected to hamper or eliminate DPPIV's chemorepellant effects.

Anti-DPPIV antibodies may be provided in any formulation, such as any pharmaceutically acceptable carrier. Due to the role of DPPIV's enzymatic activity in the regulation of many important functions as well as its role as a neutrophil chemorepellant, in most instances anti-DPPIV antibodies may be administered in a localized manner, for instance by localized injection into a tumor or in the form of a topical wound dressing.

### EXAMPLES

The following examples illustrate certain embodiments of the invention. They are not intended as a description in full detail of every aspect of the invention and should not be interpreted as such.

### Example 1

### Material Sources and Analytical Methods

For the examples herein, neutrophils were isolated from blood from healthy volunteers using Lympholyte-poly (Cedarlane Laboratories, Hornby, Canada) following the manufacturer's directions and resuspended in 2% (w/v) BSA (Fraction V, A3059, Sigma) in RPMI-1640 (Sigma).

Statistical analysis was performed using Prism (GraphPad Software, San Diego, Calif.). One-way ANOVA was used to compare between multiple groups and Student's t-test was used to compare between two groups.

### Example 2

### Neutrophil Chemotaxis Boyden Chamber Assays

Recombinant human DPPIV (“rDPPIV”) was purchased from Enzo Life Sciences (Farmingdale, N.Y.). To study transmigration of neutrophils, 50 μl of neutrophils at 2×10⁶ cells/ml in 2% (w/v) bovine serum albumin (BSA)-RPMI medium with 100 ng/ml TNF-α was added to the top chamber of a 3 μm pore size nylon membrane insert in a 24 well plate (Becton Dickinson, Franklin Lakes, N.J.) (a Boyden chamber) in the presence or absence of 100 ng/ml rDPPIV (Enzo Life Sciences) or an equal volume of 2% (w/v) BSA-RPMI. The bottom chambers contained 600 μl of 100 ng/ml TNF-α or 300 μl of 200 ng/ml rDPPIV mixed with 300 μl of 200 ng/ml TNF-60 . The transmigration was carried out for 3 hours at 37° C. in a humidified 5% CO₂ incubator. The top chamber was removed, and an aliquot of migrated neutrophils in the media of the bottom chamber was then counted with a flow cytometer. Adherent cells were stained with methylene blue and eosin (Richard-Allan Scientific, Kalamazoo, Mich.), and the number of adherent neutrophils was counted in ten different 900 μm diameter fields of view. For each experiment, counts were converted to percent of the number of migrated cells in the well with buffer in both the upper and lower chambers.

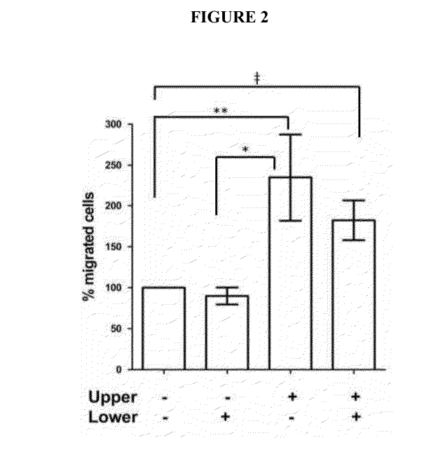

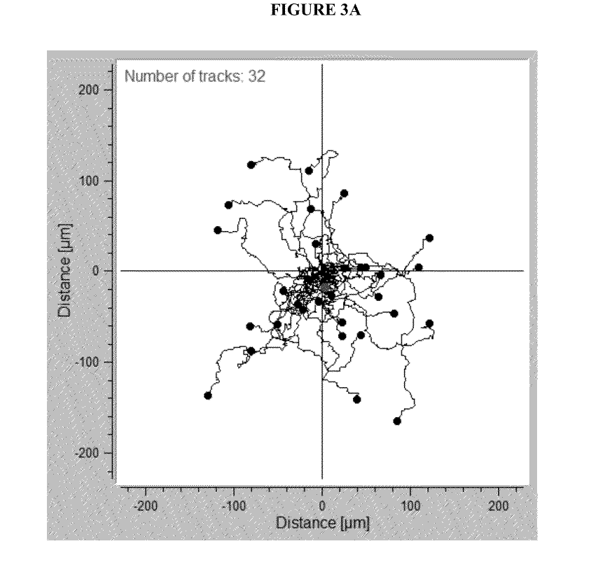

The percent of neutrophils migrating to the bottom chamber is shown in FIG. 2. When rDPPIV was added to the bottom well only, a similar amount of neutrophils migrated to the bottom well compared to the control (TNF-α buffer without rDPPIV in both the upper and lower chambers). Compared to the control and rDPPIV in the bottom well only, significantly more neutrophils migrated to the bottom well when rDPPIV was added to the top well only. When rDPPIV was added to both the top and bottom wells, significantly more cells migrated to the bottom compared to the control. Together, this data indicates that DPPIV affects human neutrophil motility.

### Example 3

### Neutrophil Chemotaxis Insall Chamber Assays

To measure the effect of rDPPIV on cell displacement using an Insall chamber (Muinonen-Martin A J, Veltman D M, Kalna G, Insall R H. An Improved Chamber for Direct Visualisation of Chemotaxis. PLoS One. 2010; 5(12):e15309), which provides a more stable gradient than a Boyden chamber, 22×22 mm glass cover slips were etched with 1 M HCl, rinsed with deionized water, and coated with 20 μg/mL bovine plasma fibronectin (Invitrogen, Life Technologies, Carlsbad, Calif.) for 30 minutes at 37° C. Cover slips were then washed twice with PBS, and neutrophils at approximately 5×10⁶ cells/ml were allowed to adhere to the coverslip for 15 minutes at 37° C. Two concentric depressions and the separating bridge in an Insall chamber slide were filled with 2% (w/v) BSA-RPMI. The media was removed from the coverslips, which were then placed face down on the chamber. Media was then removed from the outer chamber and was replaced by rDPPIV alone, DPPIV inhibitor alone (Diprotin A, Enzo Life Sciences or DPPI 1 c hydrochloride, Tocris Bioscience (Bristol, UK)), rDPPIV plus DPPIV inhibitor (all in 2% (w/v) BSA-RPMI), or 2% (w/v) BSA-RPMI. Cells located on the bridge between the square depressions were then filmed using the methods of Ochsner, S. A., et al., Disrupted function of tumor necrosis factor-alpha-stimulated gene 6 blocks cumulus cell-oocyte complex expansion. Endocrinology, 2003. 144(10): p. 4376-84, incorporated in material part by reference herein, using a 10× objective for 1 hour at 37° C. in a humidified 5% CO₂ incubator. The displacement of at least 10 randomly-chosen cells per experiment was measured over periods of 10 minutes. Cell tracking and track analysis were performed according to the methods of Phillips, J. E. and R. H. Gomer, A secreted protein is an endogenous chemorepellant in Dictyostelium discoideum. Proc Natl Acad Sci U S A, 2012. 109(27): p. 10990 - 10996, incorporated in material part by reference herein, with the exception that videos of an hour in length were processed into 10 minute segments to yield a TIFF stack of 47 images for analysis (with 13-second intervals between images).

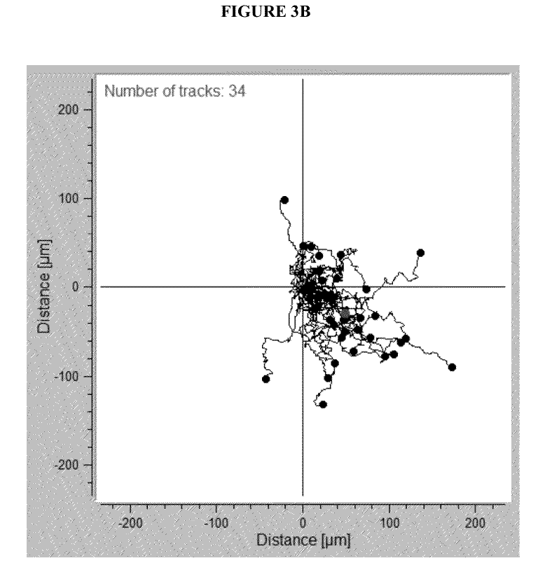

When neutrophil movement was tracked over 10 minute periods, there was no bias of movement in the media control (FIG. 3A). A biased movement away from a source of rDPPIV was observed (FIG. 3B). Cells were tracked and the average center of mass observed for the endpoints of cells in each population was determined. The center of mass of cell endpoints showed displacement away from the source of rDPPIV (FIG. 3B).

The concentration of DPPIV in human blood ranges from 400 to 800 ng/ml, or 4.7 to 9.4 nM. Therefore, we tested the ability of DPPIV to affect neutrophil migration above, below, and within this concentration range was tested using an Insall chamber and methods similar to those described above. The data from at least three independent sets of cell population tracks (one shown in FIG. 3) were analyzed to determine the forward migration index (FMI) and directionality of neutrophil migration. FMI is a measure of migration of cells along the gradient, where zero equals no movement, a positive number equals movement away from the source, and a negative number indicates movement toward the source. Directionality is the ratio of Euclidean distance to accumulated distance. For each gradient, neutrophils from at least three different volunteers were used. Results are presented in Table 1. * indicates p<0.05, ** indicates p<0.01, and *** indicates p<0.001 compared to the media control (t-test). TNF represents a gradient of rDPPIV with TNF-α stimulated neutrophils.

TABLE 1
The effects of rDPPIV on forward migration and
directness of neutrophil movement.

| | Forward Migration Index | | Directionality | |
|---|---|---|---|---|
| | Media control | | Media control | |
| rDPPIV | (0 nM | | (0 nM | |
| gradient (nM) | rDPPIV) | rDPPIV | rDPPIV) | rDPPIV |
| 0-1.2 | 0.00 ± 0.03 | 0.21 ± 0.03*** | 0.35 ± 0.02 | 0.39 ± 0.02* |
| 0-3.5 | −0.04 ± 0.04 | 0.21 ± 0.06*** | 0.26 ± 0.02 | 0.50 ± 0.03*** |
| 0-11.7 | 0.01 ± 0.06 | 0.15 ± 0.05* | 0.47 ± 0.03 | 0.47 ± 0.02 |
| 4.7-4.7 | 0.01 ± 0.06 | −0.01 ± 0.04 | 0.47 ± 0.03 | 0.30 ± 0.02*** |
| 4.7-11.7 | −0.02 ± 0.06 | 0.22 ± 0.06** | 0.50 ± 0.03 | 0.42 ± 0.04* |
| 9.4-23 | −0.01 ± 0.05 | 0.13 ± 0.05* | 0.36 ± 0.03 | 0.50 ± 0.02*** |
| 0-3.5^{TNF} | −0.02 ± 0.03 | 0.10 ± 0.03** | 0.32 ± 0.02 | 0.34 ± 0.02 |

Neutrophils showed biased movement away from higher concentrations of rDPPIV in a variety of rDPPIV concentration gradients (Table 1). An equal concentration of rDPPIV in both wells of the chamber resulted in no biased directional movement of neutrophils (Table 1). The presence of DPPIV affected directionality, although not in any systematic manner (Table 1). TNF-α stimulated neutrophils also migrated away from DPPIV (Bottom Row of Table 1). Combined, this data indicates that a gradient of DPPIV causes chemorepulsion of neutrophils.

The ability of rDPPIV to affect cell speed and directional persistence using the cell-tracking data in the Insall chamber assay was also studied. The data from at least three independent sets of cell population tracks (one shown in FIG. 3) were used to determine the average speed of neutrophils. Results are presented in Table 2. Values are mean±SEM, n=3 or more. ** indicates statistical significance compared to media control with p<0.01 by t-test.

TABLE 2
The effect of DPPIV on the average speed of neutrophils.

Average Cell Speed (μm/minute)

| | Media control | |
|---|---|---|
| rDPPIV | (0 nM | |
| gradient (nM) | rDPPIV) | rDPPIV |
| 0-1.2 | 27 ± 1 | 26 ± 1 |
| 0-3.5 | 23 ± 1 | 23 ± 1 |
| 0-11.7 | 24 ± 2 | 25 ± 2 |
| 4.7-4.7 | 21 ± 1 | 16 ± 1** |
| 4.7-11.7 | 17 ± 1 | 18 ± 2 |
| 9.4-23 | 17 ± 1 | 17 ± 1 |

A gradient of rDPPIV did not affect cell speed (Table 2). However, the addition of equal concentrations of rDPPIV on both sides of the chamber decreased cell speed (Table 2).

Furthermore, the Insall chamber assays confirm that DPPIV's chemorepulsive effects do not result from its enzymatic activity on chemoattractants. In the Insall chamber assays, neutrophils were in RPMI medium containing BSA, on a fibronectin surface. The only known enzymatic activity of DPPIV is to cleave two amino acids from the N terminus of a protein if the second amino acid is a proline or an alanine. RPMI does not contain proteins, and BSA does not have a proline or an alanine as the second amino acid. DPPIV does not cleave fibronectin. There are no known neutrophil-secreted neutrophil chemoattractants that are neutralized by DPPIV. Thus, the ability of DPPIV to cause neutrophil chemorepulsion does not appear to be caused by DPPIV's enzymatic activity on chemoattractants.

### Example 4

### Bleomycin Injury in Mouse Models

4-week-old C57/BL6 mice (Jackson, Bar Harbor, Me.) were treated with an oropharyngeal aspiration of 50 μl of saline or 3 U/kg bleomycin (Calbiochem, EMD Millipore Copr. Billerica, Mass.) in 50 μl of saline as described in Lakatos, H. F., et al., Oropharyngeal aspiration of a silica suspension produces a superior model of silicosis in the mouse when compared to intratracheal instillation. Exp Lung Res, 2006. 32(5): p. 181-99, incorporated in material part by reference herein. The successful aspiration of bleomycin into the lungs was confirmed by listening for the crackling noise heard after the aspiration. 24 hours following bleomycin aspiration (day 1), mice were treated with an oropharyngeal aspiration of 50 μl containing 0.9 μg rDPPIV (Enzo Life Sciences) in 0.9% saline or an equal volume of 0.9% saline. Mice were weighed daily, and euthanized at day 3 after bleomycin aspiration. Blood was collected by from the aorta from the euthanized mice, and serum glucose was measured using Accu-check Active (Hoffman LaRoche, Basel, Switzerland). The lungs were perfused with 300 μl of phosphate buffered saline (PBS) three times to collect cells by bronchoalveolar lavage (BAL) as described in Lakatos et al. The primary BAL cells were collected by centrifugation at 500×g for 10 minutes, and the supernatants were transferred to Eppendorf tubes. The supernatants were flash frozen with liquid nitrogen, and stored at −20° C. until further use. Primary BAL pellets were resuspended in the secondary and tertiary BAL fluid and the combined cells were collected by centrifugation at 500×g for 5 minutes. The cells were resuspended in 500 μl of 4% (w/v) BSA-PBS and counted with a hemacytometer. 100 μl of diluted cells were then aliquoted into cytospin funnels and were then spun onto glass slides (Superfrost plus white slides, VWR, Westchester, Pa.) at 400×g for 5 minutes using a cytospin centrifuge (Shandon, Cheshire, England). These cells were then air-dried, and stained with Gill's haematoxylin. Five 450 μm fields of view were counted for total cell number and the number of neutrophils as determined by observing cells with a multi-lobed nucleus. The percent of neutrophils was then multiplied by the total number of cells recovered from the BAL to obtain the number of neutrophils in the BAL.

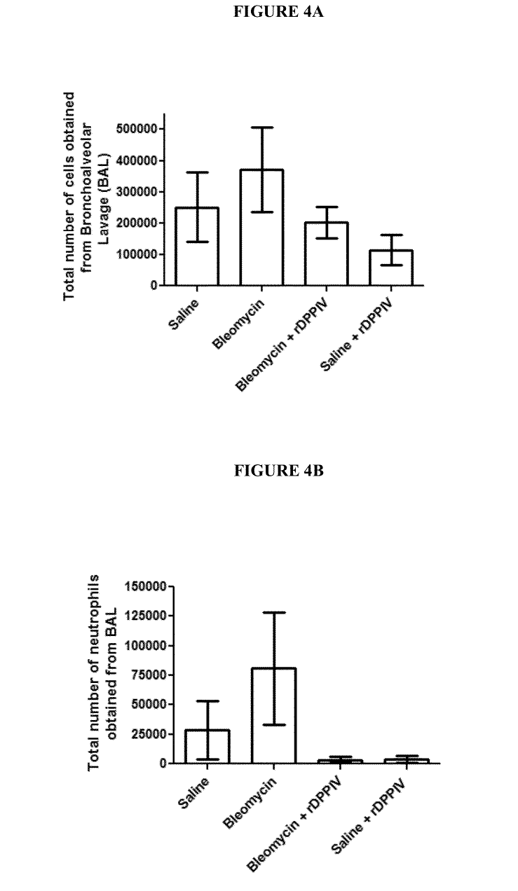

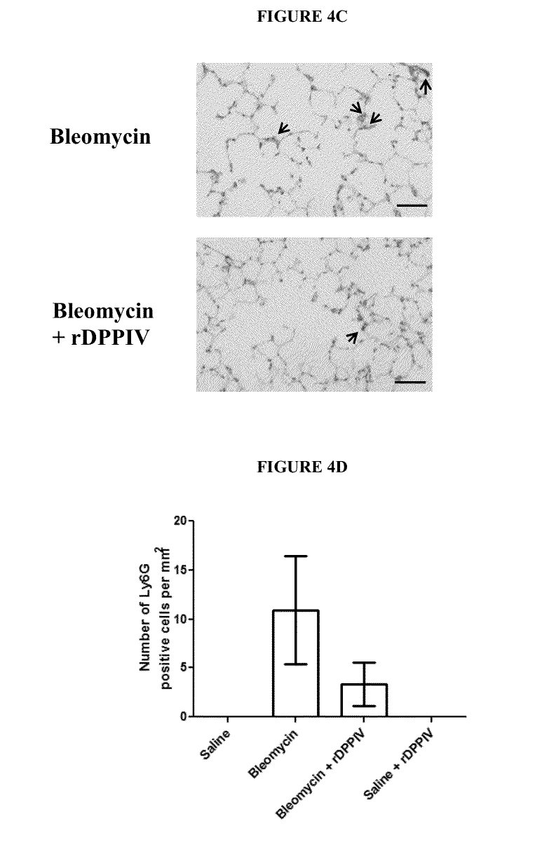

Oropharyngeal aspiration of the antibiotic bleomycin in mice causes neutrophils to accumulate in the lungs within 24 hours. At day 3, there was no statistical difference in the total number of cells from BALs of mice treated with saline, bleomycin, bleomycin and rDPPIV, or saline and rDPPIV (FIG. 4A). Cell morphology was used to determine the total number of neutrophils in the BAL. Significantly fewer neutrophils were present in the BALs from bleomycin plus rDPPIV treated mice compared to mice treated with bleomycin alone (FIG. 4B).

Additionally after BAL, lungs were inflated with pre-warmed OCT compound (VWR) and then embedded in OCT compound, frozen on dry ice, and stored at −80° C., performed as described in Pilling, D., et al., Reduction of bleomycin-induced pulmonary fibrosis by serum amyloid P. J Immunol, 2007. 179(6): p. 4035-44, incorporated in material part by reference herein. Lung tissue sections (5 μm) were prepared and immunohistochemistry was performed as described in Pilling et al., except slides were incubated with 5 μg/ml primary antibodies in 4% (w/v) BSA-PBS for 60 minutes. The lung sections were stained for Ly6G (BD Pharmingen, Franklin Lakes, N.J.) to detect neutrophils, and isotype-matched mouse irrelevant antibodies were used as controls. Slides were then washed three times with PBS over 30 minutes and incubated with 5 μg/ml biotinylated mouse F(ab′)2 anti-rat IgG in 4% (w/v) BSA-PBS for 30 minutes. Slides were then washed three times in PBS over 30 minutes and incubated with a 1:500 dilution of streptavidin alkaline phosphatase (Vector Laboratories, Ltd. UK) in 4% (w/v) BSA-PBS for 30 minutes. Staining was developed with a VectorRed Alkaline Phosphatase Kit (Vector Laboratories) for 10 minutes. Slides were then mounted as described in Pilling et al.

There were significantly fewer Ly6G positive cells in the post-BAL lungs of mice treated with bleomycin and rDPPIV compared the post-BAL lungs of mice treated with bleomycin alone (FIG. 4C and FIG. 4D). Together, the results described in FIG. 4 indicate that rDPPIV reduces the number of neutrophils in the lungs of mice.

### Example 5

### Sensitivity of Neutrophil Chemorepulsion to DPPIV Enzyme Inhibitors

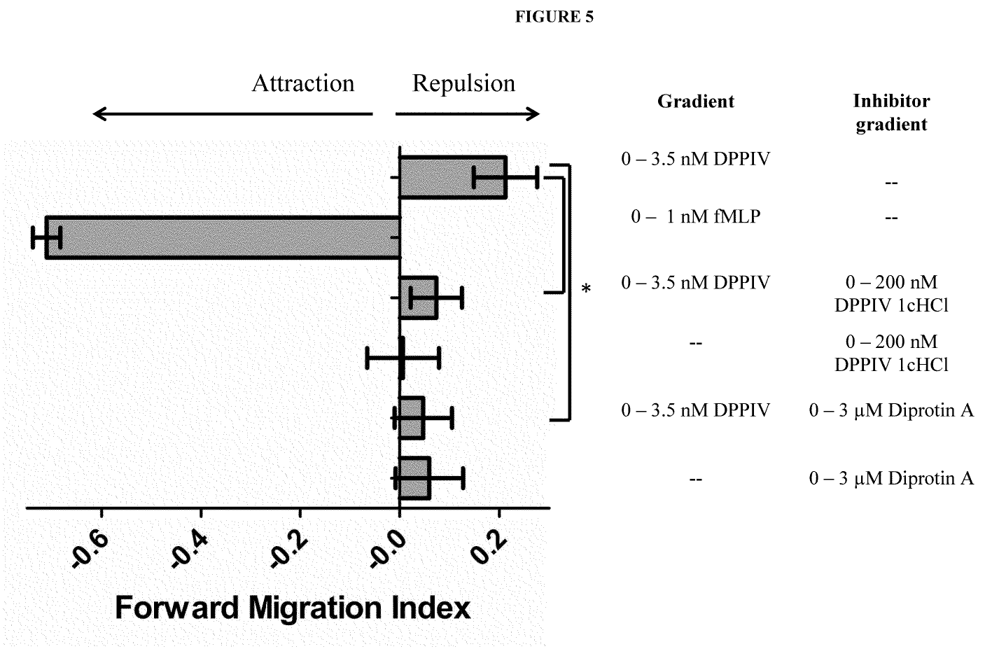

DPPIV enzyme activity has been implicated in chemotaxis of hematopoietic stem cells through cleavage of SDF-1. To determine if DPPIV enzyme activity is responsible for neutrophil chemorepulsion, two enzyme inhibitors of DPPIV, Diprotin A and DPPI 1 c HCl, were used in Insall chamber assays along with DPPIV. The inhibitors alone caused no attraction or repulsion of human neutrophils (FIG. 5). When either of the inhibitors was added with rDPPIV, the chemorepulsion of neutrophils away from the source of rDPPIV was significantly reduced compared to rDPPIV alone (FIG. 5). This suggests that the chemorepulsion of neutrophils away from DPPIV is dependent on the enzymatic activity of DPPIV.

However, such enzymatic activity likely does not exert its effects through cleavage of chemoattractants. Rather, the effect is most likely through Caveolin-1. Caveolin-1 is a protein on the surface of many cells, including neutrophils. Mutations in the active site of DPPIV that block the enzymatic activity also block the binding of DPPIV to caveolin-1. In addition, a DPPIV inhibitor also blocks the binding of DPPIV to caveolin-1. This shows that the enzymatic activity of DPPIV is necessary for the binding of DPPIV to at least one cell-surface protein. Thus, DPPIV inhibitors most likely block its chemorepulsive effect on neutrophils by interfering with binding to Caveolin-1.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details herein described, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of or “consist of the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. Further, the term “or” as used herein is intended to be inclusive, not exclusive, unless an exclusive meaning is required by context. If there is any conflict in the usages of a word or term in this specification and one or more documents that may be incorporated herein by reference, the definitions that are consistent with this specification should be adopted.

1. A method of reducing the number of neutrophils in a body region comprising administering a dipeptidyl peptidase-IV (DPPIV) composition to the body region in an amount and for a time sufficient to suppress neutrophil movement into the body region or enhancing neutrophil movement out of the body region.

2. The method according to claim 1, wherein the DPPIV comprises a portion of full-length mammalian DPPIV.

3. The method according to claim 2, wherein the portion of full-length mammalian DPPIV comprises a portion structurally homologous to a Dictyostelium autocrine proliferation repressor A (AprA) portion.

4. The method according to claim 1, wherein the DPPIV composition comprises a biodegradable polymer bound to the DPPIV.

5. The method according to claim 1, wherein the body region comprises the lungs and administering comprises administering by inhalation.

6. The method according to claim 5, wherein administering by inhalation comprises administering by nebulizer.

7. The method according to claim 1, wherein administering comprises injection.

8. The method according to claim 7, wherein the body region comprises a joint.

9. The method according to claim 1, wherein administering comprises topical administration.

10. The method according to claim 9, wherein topical administration comprises administration in the form of a cream, ointment, emollient, gel, foam, or transdermal patch.

11. The method according to claim 1, wherein the DPPIV is administered within 30 minutes of an acute injury to the body region.

12. The method according to claim 1, wherein the DPPIV is administered within 24 hours of an acute injury to the body region.

13. The method according to claim 1, wherein the DPPIV is administered at regular intervals to a body region affected by a long-term or chronic disease.

14. The method according to claim 1, wherein the DPPIV is administered in an amount of at least 33 micrograms per kilogram of body weight.

15. The method according to claim 1, wherein the DPPIV is administered in an amount of at least 3 micrograms for gram of body region weight.

16. The method according to claim 1, wherein the DPPIV is administered in an amount sufficient to generate a concentration gradient of at least 0.10 ng/ml/μm in the region.

17. A method of treating a disease, wherein at least one of the indicators of the disease is the number of neutrophils in a body region of a patient, comprising administering a dipeptidyl peptidase-IV (DPPIV) composition to the body region in an amount and for a time sufficient to suppress neutrophil movement into the body region or enhancing neutrophil movement out of the body region.

18. The method according to claim 17, wherein the disease is further defined as comprising neutrophil-mediated tissue damage.

19. The method according to claim 17, wherein the body region comprises the lungs.

20. The method according to claim 19, wherein the disease is acute respiratory distress syndrome (ARDS).

21. The method according to claim 19, wherein the disease is chronic pulmonary obstructive disease (COPD), asthma, or cystic fibrosis.

22. The method according to claim 17, wherein the body region comprises the liver.

23. The method according to claim 22, wherein the disease is alcohol-induced neutrophilic steatohepatitis or acetaminophen overdose.

24. The method according to claim 17, wherein the disease is acute phase tissue transplant rejection.

25. The method according to claim 17, wherein the disease is traumatic brain injury.

26. The method according to claim 17, wherein the body region comprises the kidneys.

27. The method according to claim 26, wherein the disease is acute glomerulonephritis or renal inflammation.

28. The method according to claim 17, wherein the body region comprises the intestines.

29. The method according to claim 28, wherein the disease is postoperative ileus, Crohn's disease, or ulcerative colitis.

30. The method according to claim 17, wherein the body region comprises the skin.

31. The method according to claim 30, wherein the disease is one of Sweet's syndrome/acute febrile neutrophilic dermatosis, rheumatoid neutrophilic dermatitis, pyoderma gangrenosum, subcorneal pustular dermatosis, Behcet's syndrome, palmoplantar pustulosis, neutrophilic eccrine hidradenitis, bowel-associated dermatosis-arthritis syndrome, or synovitis-acne-pustulosis-hyperostosis osteomyelitis (SAPHO) syndrome.

32. The method according to claim 17, wherein the disease is systemic.

33. The method according to claim 32, wherein the disease is septic shock.

34. The method according to claim 17, wherein the disease is acute pancreatitis.

35. The method according to claim 17, wherein the disease is rheumatoid arthritis.

36. The method according to claim 17, wherein the disease is one of a crystal-induced arthropathy.

37. The method according to claim 36, wherein the disease is gout.

38. The method according to claim 17, wherein the disease is a reperfusion injury.

39. The method according to claim 38, wherein the disease is one of pressure ulcer, diabetic foot ulcer, myocardial infarction, stroke, ischemic brain injury, or ischemic bowel disease.
